(19) United States
(12) Patent Application Publication
(10) Pub. No.: US 2007/0254837 A1
(43) Pub. Date: Nov. 1, 2007

(54) Treatment for Necrotizing Enterocolitis

(75) Inventors: James A. Hardin, Calgary (CA); Andre Gerald Buret, Calgary (CA)

Correspondence Address: TORYS LLP, 79 WELLINGTON ST. WEST, SUITE 3000, TORONTO, ON, M5K 1N2, CA

(73) Assignee: UNIVERSITY TECHNOLOGIES INTERNATIONAL INC., Alberta (CA)

(21) Appl. No.: 10/596,565

(22) PCT Filed: Dec. 16, 2004
(86) PCT No.: PCT/CA04/02142

§ 371 Date: May 7, 2007

Related U.S. Application Data
(60) Provisional application No. 60/529,574, filed on Dec. 16, 2003.

(51) Int. Cl. A61K 38/18; A61K 31/04; A61K 31/195; A61K 31/198; A61P 1/00; A61K 31/295; A61K 31/34

(52) U.S. Cl. 514/12; 514/470; 514/502; 514/562; 514/565; 514/645

(57) ABSTRACT

The invention provides compositions and methods for the treatment of necrotizing enterocolitis (NEC). A treatment of epidermal growth factor (EGF) receptor agonist and L-arginine, a bioequivalent thereof, or NO-donor showed efficacy against NEC. The invention also provides kits, unit doses and uses comprising epidermal growth factor (EGF) receptor agonist and L-arginine, a bioequivalent thereof, or NO-donor for the treatment of NEC.

### FIELD OF THE INVENTION

This invention relates generally to compositions and methods for treating or preventing necrotizing enterocolitis (NEC).

### BACKGROUND OF THE INVENTION

Necrotizing enterocolitis is a devastating illness in premature infants. The pathogenesis of NEC involves a combination of predisposing factors that leads to mucosal injury and intestinal necrosis. Intestinal ischemia, enteral feeding, bacterial colonization, and gut immaturity have all been implicated in the pathogenesis of NEC (17;20). NEC is rarely, if ever, observed in utero and ninety percent of infants with NEC are born preterm (20) making premature birth the single most common risk factor for the condition in humans. Amniotic fluid contains hormones and peptides that play a role in intestinal maturation and preparation for postnatal enteral feeding. Preterm birth may not allow for proper maturation of the gut. Furthermore, a 6-10 fold increase in the incidence of NEC has been reported in formula-fed infants compared with breast-fed infants (20).

Recent studies have reported a significant decrease in salivary and circulating epidermal growth factor (EGF) in infants with NEC suggesting a relationship between reduced levels of EGF and the development of NEC (44). EGF is present in breast milk, and studies have shown NEC occurs less often in premature infants fed breast milk compared with formula-fed infants (43). However, in the clinical setting, these patients are intubated. As feeding of human milk in premature infants of less than 1500 g has been associated with poorer rates of growth and nutritional deficits (43), most of their nutritional requirements are met via total parenteral nutrition administered through a central line. Furthermore, technical factors associated with the collection, storage and delivery of breast milk to premature infants renders the use of human breast milk difficult, and even unlikely in this clinical context.

Anti-infective properties of EGF against a variety of pathogens have been previously demonstrated (6-12;29). Bacterial colonization has been identified as a prerequisite in the development of NEC (17). Although NEC can present in clusters (17) and displays an epidemiology reminiscent of a nosocomial infection (3;30) no particular pathogen has been associated with the pathogenesis of NEC. These findings suggest that NEC may be the result of a secondary inflammatory response to the colonizing organisms rather than a direct infection. The role of bacterial colonization in the development of necrotizing enterocolitis is supported by evidence indicating that oral antibiotics reduce the incidence of NEC in low birth weight infants (13). EGF enhances mucosal wound repair.

This effect appears to be due to both a direct effect of EGF to accelerate mucosal wound repair (27;41) and to the anti-infective properties of the peptide. Experimental gastric ulcers are rapidly colonized by various bacteria, resulting in delayed healing. Both antibiotics and EGF treatment accelerated ulcer healing in parallel with reduced bacterial colonization (25;26). In addition, EGF has been shown to enhance gut maturation in neonates (38;39). Epidermal growth factor has also been shown to prevent gastric mucosal injury induced by necrotizing agents such as absolute ethanol and aspirin. This mucosal protection conferred by EGF in the stomach is accompanied by an increase in gastric blood flow (33).

Intestinal ischemia has been implicated as a risk factor in the development of NEC. Nitric oxide (NO) is a biological mediator that is produced by the activity of the enzyme nitric oxide synthase on the amino acid L-arginine. In the gastrointestinal system, NO is an important regulator of mucosal blood flow. In the face of injury or inflammation, NO is critical to the maintenance of mucosal integrity and intestinal barrier function. Inhibition of NO synthesis in a variety of animal models of bowel injury is associated with increased intestinal damage (16;36). Application of exogenous sources of NO in these models attenuates the damage. In a neonatal piglet model of NEC, infusion with L-arginine markedly reduced intestinal injury (22). Furthermore, plasma L-arginine concentrations are decreased in premature infants diagnosed with NEC suggesting a causal relationship (47). In a recent double-blind, placebo controlled trial conducted with a population of 152 infants (birthweight ≦1250 g and gestational age ≦32 weeks), dietary L-arginine supplementation significantly reduced the incidence of NEC in high risk neonates from 27% to 7% (2), clearly demonstrating therapeutic efficacy.

Other factors have been found to have a beneficial effect against NEC, for example: breast milk (23;37); L-carnitine (1) , platelet-activating factor receptor antagonists (15), intestinal Lactobacillus and Bifidobacterial supplementation (14;32); interleukin 10 (40), IgA supplementation (24), enteral antibiotic prophylaxis (45), early postnatal dexamethasone treatment (28), and neonatal formula supplemented with egg phospholipids (19).

Despite the many agents known to have potential in the treatment of NEC, in the clinical setting, the most common treatment for NEC remains a regimen of antibiotics. Systemic antibiotic therapy with ampicillin and gentamicin is typically provided unless resistant Staphylococcus epidermidis is suspected, in which case vancomycin is used instead of ampicillin. Clinamycin, metronidazole, or other anaerobic therapy is often used to treat anaerobic infections if perforation is suspected or has occurred (35). There is, however, no fully preventative or therapeutic treatment for preventing or treating NEC.

### SUMMARY OF THE INVENTION

An aspect of the invention is to provide a composition comprising an EGF receptor agonist and L-arginine, a bioequivalent of L-arginine, or an NO-donor wherein the ratio of the agonist and L-arginine, bioequivalent thereof, or NO-donor is between 1:45,000,000 and 1:4,500 (mole:mole), or between 1:20,000,000 and 1:100,000, or between 1:10,000,000 and 1:1,000,000, or between 1:4,700,000 and 1:47,000. The composition can be solid, lyophilized or solution form. The composition can be delivered by orally or enterally. The EGF receptor agonist can be EGF. The composition can be used for the treatment of necrotizing enterocolitis.

Another aspect of the invention is to provide a unit dose comprising L-arginine, bioequivalent thereof, or an NO-donor and an EGF receptor agonist suitable for the oral administration to an animal upon dissolution with a pharmaceutically acceptable liquid. The pharmaceutically acceptable liquid is selected from the group consisting of water, saline, infant formula, buffered solution, expressed breast milk, other suitable carriers, and combinations thereof. The unit dose may comprise L-arginine in a quantity of from about 200 mg/kg/day (0.9 mmol/kg/day) to about 500 mg/kg/day (2.4 mmol/kg/day), or more preferably from about 250 mg/kg/day (1.2 mmol/kg/day) to about 400 mg/kg/day (1.9 mmol/kg/day), or more preferably from about 300 mg/kg/day (1.4 mmol/kg/day) to about 350 mg/kg/day (1.6 mmol/kg/day). The EGF receptor agonist may be supplied in a quantity of 0.032 nmol/kg/day to about 0.32 umol/kg/day, or more preferably from about 0.16 nmol/kg/day to about 0.16 mmol/kg/day, or more preferably from about 0.32 nmol/kg/day to about 32 nmol/kg/day.

Another aspect of the invention is to provide a unit dose comprising L-arginine, a bioequivalent thereof, or an NO-donor and an EGF receptor agonist suitable for the intravenous administration to a human infant, optionally upon dissolution with a pharmaceutically suitable solution. The ratio of the agonist and L-arginine, bioequivalent thereof, or NO-donor may be between 1:45,000,000 and 1:4,500 (mole:mole), or between 1:20,000,000 and 1:100,000, or between 1:10,000,000 and 1:1,000,000, or between 1:4,700,000 and 1:47,000.

Another aspect of the invention is to provide a method of treating necrotizing enterocolitis in an animal and a method of prophylaxis of necrotizing enterocolitis in an animal, the method comprising administering an EGF receptor agonist and L-arginine, a bioequivalent thereof, or an NO-donor to the animal. The method may be used to treat an infant, particularly an infant suffering from cardiovascular disturbances, or a premature infant at an age prior to normal term. For example, the human infant may have a weight of less than or equal to about 1700 g, or less than about 1400 g, more preferably less than about 1300 g, more preferably less than about 1200 g, more preferably less than about 1100 g, more preferably less than about 1000 g, more preferably less than about 900 g, more preferably less than about 800 g, more preferably less than about 750 g.

Another aspect of the invention is to provide a method of treating necrotizing enterocolitis or of prophylaxis of necrotizing enterocolitis in a premature infant, the method comprising enterally administering to the infant an EGF receptor agonist and L-arginine, a bioequivalent thereof, or an NO-donor. The EGF receptor agonist and L-arginine, a bioequivalent thereof, or an NO-donor may be administered together in a mixture. The mixture may be administered at least once daily.

Another aspect of the invention is to provide a kit comprising therapeutic amounts of an EGF receptor agonist and L-arginine, a bioequivalent thereof, or an NO-donor, and instructions for use in the treatment of a medical disorder, for example necrotizing enterocolitis. The agonist and L-arginine, a bioequivalent thereof, or NO-donor may be supplied combined in solid form. The instructions may include the step of dissolving the solid form in a solution suitable for oral administration or intravenous administration. The agonist and L-arginine, a bioequivalent thereof, or an NO-donor may be supplied separately. The instructions may include a step of mixing the agonist and L-arginine, a bioequivalent thereof, or an NO-donor before administration.

Another aspect of the invention is to provide a method of treating NEC in an animal comprising delivering an EGF receptor agonist to the intestinal tract of the animal and increasing the in vivo generation of NO within the intestinal tract of the animal. This can be done by administering a substrate of nitric oxide synthase, a bioequivalent of the substrate, or an NO donor to the animal.

Another aspect of the invention is to provide a method of treating a person, optionally an infant, at risk of NEC comprising delivering an EGF receptor agonist to the intestinal tract of the patient and increasing the in vivo generation of NO within the intestinal tract of the person. This can be done by administering a substrate of nitric oxide synthase, a bioequivalent of the substrate, or an NO donor to the animal.

Another aspect of the invention is to provide a use of an EGF receptor agonist and L-arginine, a bioequivalent thereof, or an NO-donor for treating necrotizing enterocolitis in an animal. The animal may be an infant, particularly an infant suffering from cardiovascular disturbances, or a premature infant at an age prior to normal term. For example, the infant may have a weight of less than or equal to about 1700 g, or less than about 1400 g, more preferably less than about 1300 g, more preferably less than about 1200 g, more preferably less than about 1100 g, more preferably less than about 1000 g, more preferably less than about 900 g, more preferably less than about 800 g, more preferably less than about 750 g. The use can be for the prophylaxis of necrotizing enterocolitis in a premature infant. The EGF receptor agonist and L-arginine, a bioequivalent thereof, or an NO-donor may be administered together in a mixture. For example, the mixture may be administered at least once daily.

Another aspect of the invention is to provide a use of an EGF receptor agonist and a second component, the second component capable of increasing the in vivo generation of NO within the intestinal tract of the animal, for treating NEC in an animal. For example, the second component may be a substrate of nitric oxide synthase, or a bioequivalent of the substrate, or an NO-donor.

Another aspect of the invention is to provide a use of an EGF receptor agonist and a second component, the second component capable of increasing the in vivo generation of NO within the intestinal tract of a person, for treating a person, optionally an infant at risk of NEC. For example, the second component may be a substrate of nitric oxide synthase, or a bioequivalent of the substrate, or an NO-donor.

These and other aspects of the present invention will become evident upon reference to the following detailed description and attached drawings. In addition, various references are set forth below in the “list of references” on pages 13 to 18 which describe in more detail certain procedures, devices or compositions. All references on pages 13 to 18 are incorporated herein by reference as though each document were reproduced herein in its entirety. Applicant reserves the right, at its discretion, to incorporate directly any or all references on pages 13 to 18 during pendency of this application.

### BRIEF DESCRIPTION OF THE DRAWINGS

FIGS. 1A to 1E are representative photomicrographs of small bowel from (1A) control, (1B) untreated NEC, (1C) L-arginine-treated, (1D) EGF-treated, and (1E) L-arginine+EGF-treated animals. Arrows on the untreated panel indicate area of discoloration and distension (left arrow) and stenosis (right arrow), respectively;

FIG. 2 shows clinical scoring on autopsy in the control (n=10), untreated (NEC, n=12), L-arginine-treated (n=14), EGF-treated (n=15), and EGF+L-arginine -treated animals (n=15);

FIG. 3 shows a breakdown of individual parameters used in the clinical scoring following autopsy in control (n=10), untreated (NEC, n=12), L-arginine-treated (n=14), EGF-treated (n=15), and EGF+L-arginine-treated animals (n=15);

FIGS. 4A to 4E are representative photomicrographs of distal ileal tissue sections obtained from (4A) control, (4B) untreated NEC, (4C) L-arginine-treated, (4D) EGF-treated, and (4E) L-arginine+EGF-treated animals;

FIG. 5 shows damage scores in distal ileal tissue obtained from control (n=10), untreated (NEC, n=15), L-arginine-treated (n=11), EGF-treated (n=14), and EGF+L-arginine-treated animals (n=14); and

FIG. 6 shows total lactase activity in distal ileal tissue obtained from control (n=18), untreated (NEC, n=11), L-arginine-treated (n=11), EGF-treated (n=14), and EGF+L-arginine-treated animals (n=10).

### DETAILED DESCRIPTION OF THE INVENTION

Prior to setting forth the invention, it may be helpful to an understanding thereof to set forth definitions of certain terms that will be used hereinafter.

“Necrotizing enterocolitis” (NEC) as used herein means a gastrointestinal disease characterized by: systemic symptoms, ranging from apnea, bradycardia and temperature instability to diffuse intravascular coagulation and septic shock, intestinal symptoms such as abdominal distension and bloody stools and radiological findings such as pneumatosis intestinalis and gas in the portal vein.

“EGF receptor agonist” as used herein means any molecule which will produce a biochemical effect when bound to any of the erbB(1-4) receptors, particularly the erbB1 receptor, such that any or all of the following effects occur: intestinal glucose transport is increased, the apical surface of the enterocytes (cells lining the lumen of the small intestine) are altered, the colonization and translocation of pathogenic organisms across mucosal surfaces is inhibited, and gut maturation is induced. The molecule is preferably epidermal growth factor. It may otherwise be an antibody, small molecule, protein, peptide, peptidic analogues, or peptidomimetic.

“Epidermal growth factor” or EGF as used herein is a 53-amino acid protein known to be synthesized in the duodenum and salivary glands of normal humans, and expressed in human breast milk. The amino acid sequence of human EGF is:

(SEQ ID NO: 1)

Asn Ser Asp Ser Glu Cys Pro Leu Ser His Asp Gly
Tyr Cys Leu His Asp Gly Val Cys Met Tyr Ile Glu
Ala Leu Asp Lys Tyr Ala Cys Asn Cys Val Val Gly
Tyr Ile Gly Glu Arg Cys Gln Tyr Arg Asp Leu Lys
Trp Trp Glu Leu Arg.

The protein used in the experiments described herein had the foregoing sequence. Non-human EGF sequences which act as EGF in humans are also contemplated. Species variants of EGF are thus also included, such as described for mouse, rat and pig (49-52), or bovine EGF as cited in U.S. patent application 20030059802, or so-called supra-agonistic chimeras of different EGF receptor ligands (53). This definition also refers to a polypeptide having substantially the same sequence and activity as purified native epidermal growth factor. This includes recombinantly and chemically synthesized peptides or proteins. This term also refers to proteins varying from the native sequence by substitution with other amino acids or deletion of one or more amino acids, as long as the EGF biological activity is substantially preserved. The definition also includes fragments, peptidic analogues, and peptidomimetics of EGF as long as the EGF biological activity is substantially preserved. EGF biological activity can be screened by a receptor binding assay, and confirmed using any of the methods indicated above in connection with receptor agonists. Thus, for example, a human EGF protein in which the methionine (Met) at position 21 is replaced with isoleucine (Ile) falls within the scope of “EGF.” Such a protein is denoted hEGF-I₂₁ generally, and is generally denoted rhEGF-I₂₁ if prepared recombinantly (chemically synthesized hEGF is included in the term “hEGF”). Similarly, hEGF having the Asp at position 11 replaced with Glu is generally denoted hEGF-E₁₁. Some EGF proteins truncated near the carboxy terminal retain their biological activity, and are generally denoted with a subscript indicating the last peptide residue retained. Thus, EGF lacking the last 2 of its normal 53 peptides is generally indicated as EGF₅₁. Proteins having an amino acid deletion, for example wherein Trp₄₉ is absent, are generally denoted with the term “del” (or .DELTA.) and a subscript indicating the position, without altering the numbering of the remaining amino acids. Thus, if Trp₄₉ were deleted, the resulting protein would be indicated EGF-.DELTA.₄₉. Insertions, increasing the chain length, are generally indicated as substitutions substituting 2 or more amino acids for one, e.g., rhEGF-L/G₁₅ indicates insertion of Gly after the natural Leu₁₅. Finally, an EGF of the invention where His₁₆ has been replaced by another amino acid, with or without other modifications, is generally denoted generically by EGF-X₁₆. Muteins of EGF, as described for example in U.S. Pat. No. 6,191,106 (Mullenbach et al.), which issued Feb. 20, 2001 also fall within this definition provided they have the requisite EGF activity.

“L-Arginine” as used herein means the semiessential amino acid (2-amino-5-guanidinovaleric acid) and its salts, e.g., acid addition salts suitable for administration to a mammal. A bioequivalent of L-arginine is a compound which, like L-arginine, is a substrate of nitric oxide synthase which generates NO in vivo, or may be converted to a substrate of nitric oxide synthase such as L-citrulline via the arginine-citrulline cycle or enzymes of the urea cycle. The rate-limiting enzyme in endogenous L-arginine production is argininosuccinate synthase. The main site of endogenous L-arginine production is the kidney, which converts L-citrulline to L-arginine (59). Glutamine is converted to L-citrulline in the small intestine (63), and ornithine alpha-ketoglutarate is a precursor of glutamine (61). A compound, typically a small organic molecule, capable of donating an NO molecule, an “NO donor” when administered in vivo may also be administered. Such compounds include, but are not limited to, S-nitroso-N-acetyl-penicillamine (SNAP), 3-morpholinosydnonimine (SIN-1), sodium nitroprusside (SNP) 4-phenyl-3-furoxancarbonitrile (PFC), glyceryl trinitrate (GTN), and isosorbide dinitrate (ISDN) (60, 61, 62). NO production can be measured in vitro by the Griess assay (54), or by chemiluminescent detection (55, 56), or in vivo by the use of manometry and electronic nitric oxide sensors (57, 58). A commercial assay for in vitro detection of NO is available from Cayman Chemicals (Ann Arbor, Mich.).

The present invention includes a treatment for necrotizing enterocolitis. The subject of the treatment may already be suffering from the condition as indicated by any one or more of the following symptoms: abdominal distension and tenderness, pneumatosis intestinalis, gas in the portal vein, occult or frank blood in the stools, intestinal gangrene, bowel perforation, apnea, bradycardia, temperature instability, diffuse intravascular coagulation, sepsis and shock (35). The subject may be at risk for the condition. Such a subject includes full term infants that may be admitted to the neonatal intensive care unit for other reasons such as cyanotic heart disease, enteritis polycythemia or birth asphyxia , premature infants, i.e. an infant born before the 37^{th} week of gestation, or a very low weight birth weight infant, one of less than about 1500 g (incidence rises to about 10% in infants less than 1500 g (17)), down to as little 750 g or less, or even previously healthy full term infants in well baby nurseries (4;46).

Treatment or Prevention of NEC

Treatment or prevention of NEC involves an EGF receptor agonist, and L-arginine or a bioequivalent thereof. A treatment of the invention is generally administered enterally. If this route is contraindicated, the treatment may be administered intravenously.

In a preferred embodiment, the invention is an oral product, possibly a pharmaceutical, for treating NEC. The product can be dietary product, or the active ingredients can be provided in a form suitable for addition to a dietary product, e.g., milk water, saline, buffered solutions, infant formula, and expressed breast milk, other suitable carriers, or combinations thereof. The active ingredients can then be mixed to the dietary product just prior to administration.

In a preferred embodiment, the invention is a product that includes both an EGF receptor agonist and L-arginine as a therapeutic for the prevention and/or treatment of necrotizing enterocolitis in infants. Experiments performed, which establish the feasibility of this combination of such agents as an effective treatment, are described below.

Materials and Methods

Animal Model of Disease

These studies utilized a well-validated neonatal rat model of necrotizing enterocolitis (14). Briefly, neonatal Sprague-Dawley rats (Charles River Laboratories Inc., Wilmington, Mass.) were collected from their mothers immediately after birth, prior to suckling maternal milk. Animals were weighed and placed in infant incubators to control body temperature. Subsequently, rats were hand fed via the orogastric route 0.1 ml of rat milk substitute (RMS) consisting of prepared infant formula (Esbilac formula, Pet-Ag, New Hampshire, Ill.) plus or minus the therapeutic supplement every three hours. EGF (Austral Biologics, San Ramon, Calif.) was produced by genetically engineered yeast and purified by sequential chromatography and reverse phase high pressure liquid chromatography (HPLC) and determined to be >97% pure by N-terminal amino acid sequencing, amino acid composition, HPLC analysis and SDS gel electrophoresis. Arginine (L-arginine hydrochloride, crystalline, ICN Biomedicals Inc, Aurora, Ohio) was produced by a proprietary synthetic process. EGF (100 ng/ml) and L-arginine (1.35-2.7 mg/ml) were admininstered based on daily feed intake to achieve a dose of 1.5 mmol/kg/day of L-arginine (in standardized units these doses work out to ˜20 ug EGF/kg/day and ˜316 mg L-arginine/kg/day, for a ratio of about 1:472 500 mole/mole). The EGF and L-arginine were gently vortexed and thoroughly mixed into the Esbilac infant formula. After 48 hrs of feeding, volumes were advanced slowly to 0.3 ml as tolerated. To induce clinical disease rats were stressed twice daily with asphyxia by breathing 100% nitrogen gas for 60 sec followed by cold stress at 4° C. for 10 min starting one hour after birth.

Experimental Design

Six separate experimental groups were studied: unstressed rats artificially fed with EGF-free rat milk substitute (RMS) (Control, n=10); stressed rats that were dam-fed (Control (s), n=7); stressed rats artificially fed EGF-free RMS (NEC, n=10); stressed rats artificially fed RMS supplemented with 100 ng/ml recombinant human EGF (EGF-NEC, n=6); stressed rats artificially fed RMS supplemented with 1.5 mmol L-arginine/kg/day (ARG-NEC, n=6); and stressed rats artificially fed RMS supplemented with 100 ng/ml recombinant human EGF+1.5 mmol L-arginine/kg/day (EGF/ARG-NEC, n=8). Animals that developed abdominal distension, respiratory distress or lethargy during the 96-hour course of the experiment were terminated. After 96 hours all surviving animals were killed by decapitation and the small intestine removed for further analysis.

Measurements

Body weights and tail lengths were recorded daily, and twice daily urination and defecation was induced by gentle stimulation of the anogenital region and stool collected for future analysis. The small intestine was visually assessed for clinical signs of NEC such as intestinal hemorrhage and discoloration, and ileal distension and stenosis. The small intestine was then divided into two halves and 2-3 cm of tissue from the distal segment (ileum) fixed, embedded in paraffin, and counterstained with hematoxylin and eosin for histological evaluation of NEC (14). Histology was evaluated in a blinded manner and graded as follows: normal (0) showing no damage; mild (+1) showing slight submucosal and/or lamina propria separation (+2); moderate (+2) displaying moderate separation of the submucosa and/or lamina propria, and/or edema in the submucosal and muscular layers; severe (+3) showing severe separation of submucosa and/or lamina propria, and/or severe edema in the submucosal and muscular layers, and regional villus sloughing; necrosis (+4) displaying loss of villi and necrosis (14). The length of the remaining ileum was measured and the tissue homogenized in 2.5 mM ethylenediaminetetraacetic acid (EDTA), snap frozen in liquid nitrogen, and stored at 70° C. for later analysis of lactase activity (21) and ileal protein (5) and DNA (3 1) content.

Statistical Analysis

Data are expressed as means ±SE. Data analysis was performed by analysis of variance (ANOVA) followed by Tukey's posthoc multiple comparison test. Significance levels were set at 0.05.

Results

Clinical Assessment

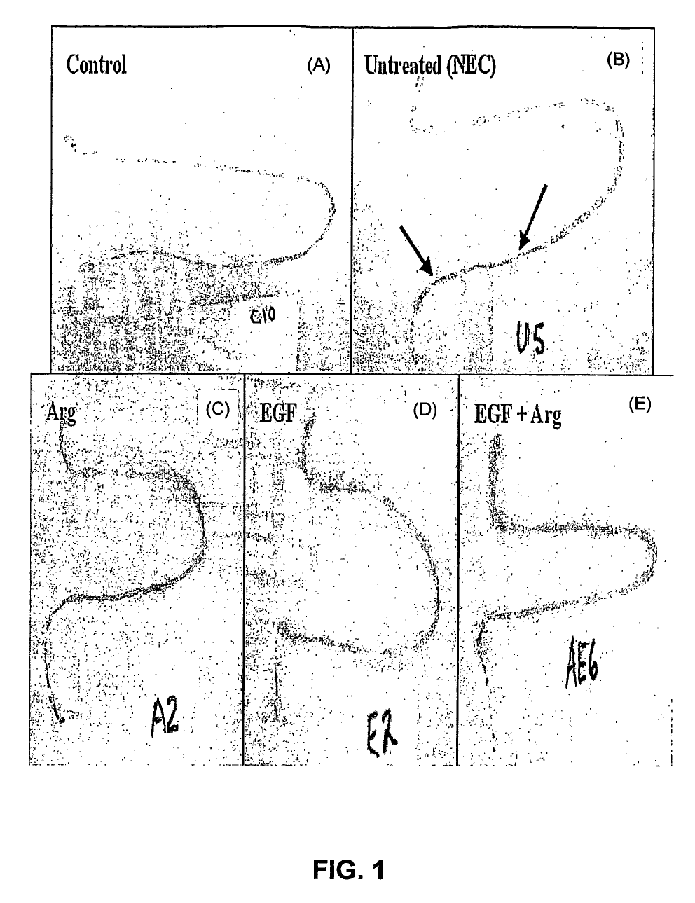

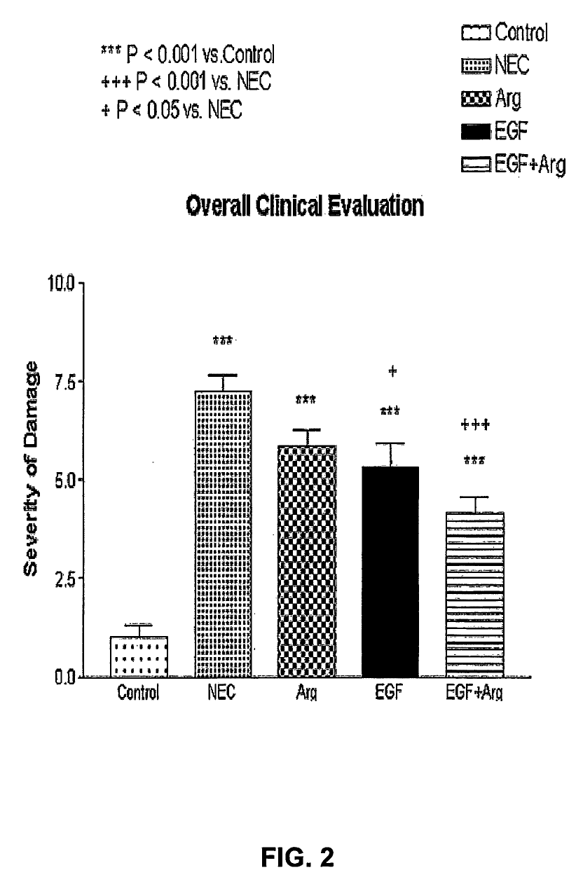

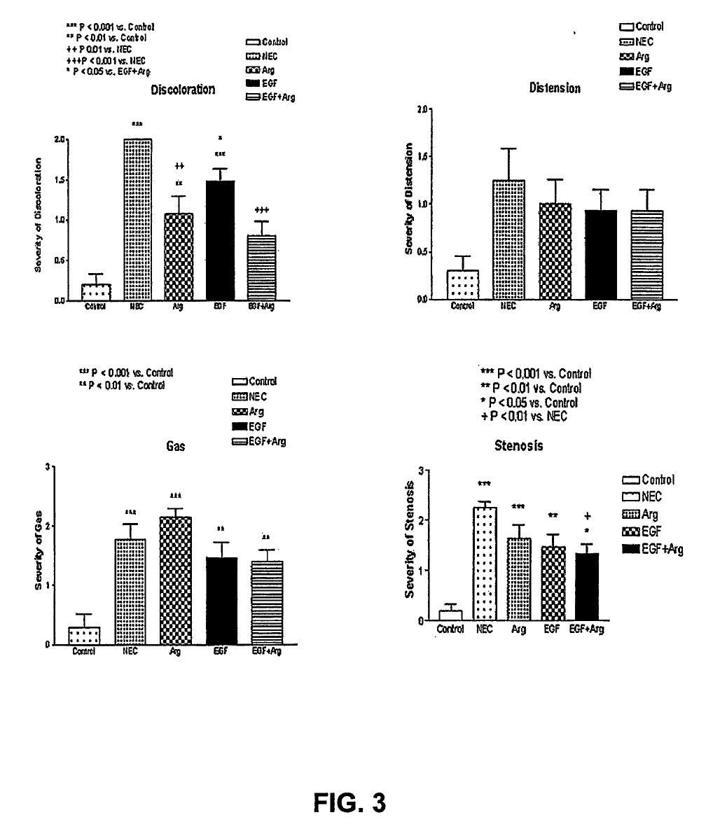

Representative photomicrographs of small bowel obtained from control, untreated, Arg, EGF and Arg+EGF animals are shown in FIG. 1. Macroscopic examination of the GI tract on autopsy showed clear evidence of intestinal damage in the stressed untreated group similar to that seen in human neonatal NEC. Evidence of inflammation and hemorrhage was noted, and the ileum was discolored with prominent distension and stenosis. In animals undergoing treatment and displaying a more moderate progression of the disease, pathological changes in the small intestine were patchier, with scattered areas of distension, stenosis and hemorrhage. The overall clinical appearance of each animal was assessed (FIG. 2). Untreated animals displayed significant tissue pathology over that seen in controls. Severity of damage did not differ between the untreated or the Arg-treated groups. In contrast, both the EGF and EGF+Arg treatment groups showed significantly less pathology and damage scores in these groups were significantly less than that observed in the untreated (NEC) group though still significantly increased over control animals. The separate parameters examined in the clinical assessment are summarized in FIG. 3. Discoloration, a visual assessment of luminal blood, was significantly elevated in the untreated, Arg and EGF-treated groups compared to controls. Discoloration did not differ between control animals and those in the EGF+Arg-treated group. Both Arg and EGF+Arg treated groups showed significantly less discoloration than observed in the untreated group. While ileal distension was increased in all groups compared to controls none of the values reached statistical significance. Luminal gas was elevated in all groups compared to controls. Luminal gas did not differ between any of the treatment groups and the untreated (NEC) group. The untreated, Arg, EGF, and EGF+Arg-treated groups all showed significant stenosis when compared to controls. Stenosis in the EGF+Arg treated group was significantly less than that seen in the untreated (NEC) group.

Histology

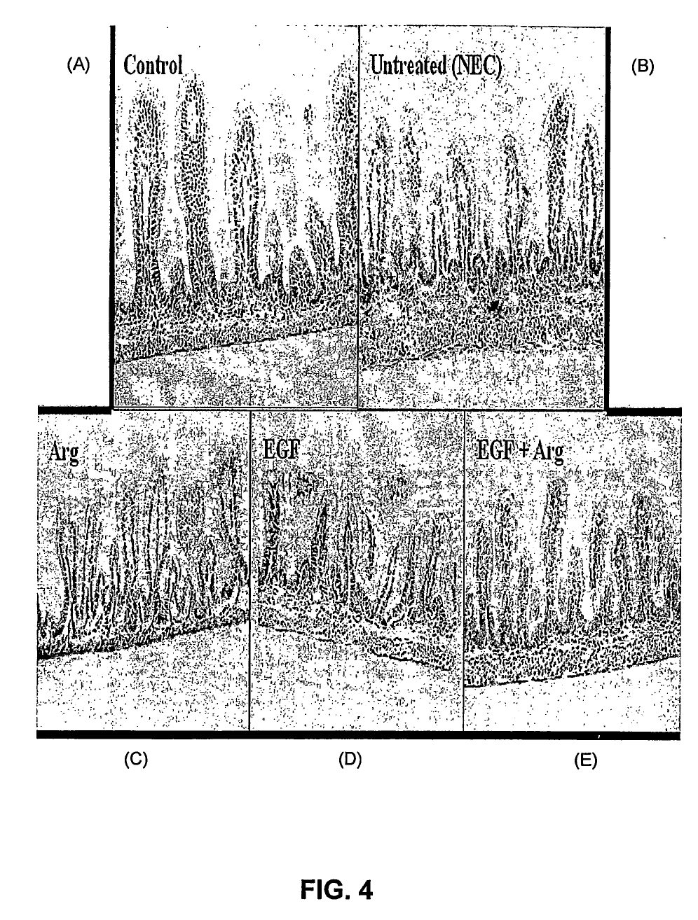

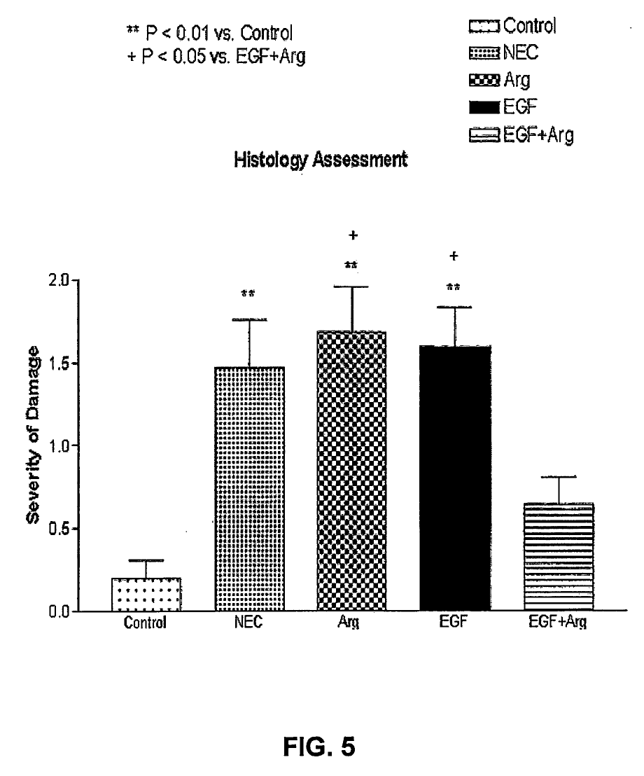

Representative photomicrographs of ileal tissue sections from control, untreated, Arg, EGF, and EGF+Arg-treated animals are shown in FIG. 4. Extensive edema, epithelial delamination and submucosal separation were all prominent features of tissue obtained from the untreated group. Significant damage was also observed in both the Arg and the EGF-treatment group while tissue obtained from animals receiving the combined EGF+Arg treatment did not appear much different from controls. Histological analysis of ileal tissue was performed and is shown in FIG. 5. In the untreated (NEC) group microscopic examination identified significant pathological changes in ileal morphology and structure. Histological damage scores did not differ between NEC animals and those treated with either EGF or L-arginine alone. Combined EGF/L-arginine treatment reduced the histological damage associated with the disease such that histological scores did not differ from controls. Histological damage in the EGF+Arg group was significantly less than that measured in the Arg or EGF-treatment groups.

Lactase Activity

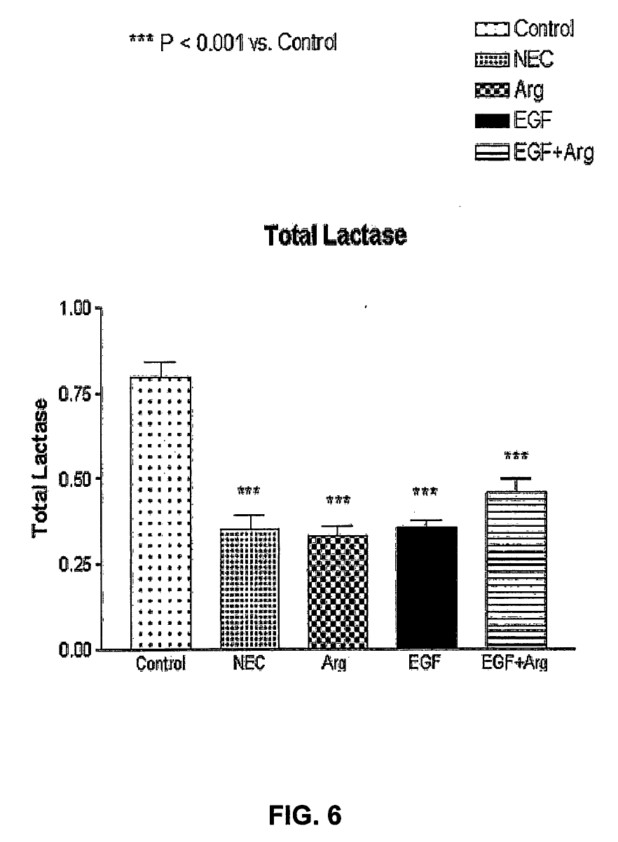

Additional measurements assessed the functional integrity of the small intestine by assaying the activity of the digestive enzyme lactase (FIG. 6). Total lactase activity was significantly decreased in NEC rats compared to controls. EGF or L-arginine treatment alone did not alter the deficit in lactase activity. In stressed animals treated with combined EGF/L-arginine therapy, total lactase activity was increased over the untreated group and the Arg or EGF-treated animals but this effect did not reach statistical significance.

Conclusions

The foregoing results thus establish that combined EGF-L-arginine treatment can provide clinical benefits and efficaciousness superior to those that would be expected from the previously known effects of their individual administration. While EGF alone was shown to provide some protection against physical damage to the intestine and L-arginine alone decreased the appearance of blood in the lumen of sick animals, only the combined EGF-L-arginine treatment improved both measurements. A combined treatment of EGF-L-arginine reduced the level of overall damage and injury in an established model of necrotizing enterocolitis.

The relative amounts of EGF receptor agonist and L-arginine are conveniently determined on a molar basis. A bioequivalent of L-arginine, if capable of producing, on a molar basis, a greater amount of NO than L-arginine as a substrate of nitric oxide synthase, would be determined on a mole-equivalent basis of the amount of NO the bioequivalent is capable of producing. Similarly, the amount of NO released by an NO-donor would be determined on a mole-equivalent basis.

A treatment of the present invention, for administration to an infant, would require administration by an appropriate route. As the agents are to preferably be administered so as to be biologically available to the free luminal side of epithelial cells of the intestine, oral administration would be most preferred. As such, a unit dose of the two agents would be provided in a suitable container for ready opening and delivery to and mixture with a dietary product. A suitable amount of the components of the present invention could thus be provided in a powdered or granular form to be added directly to milk or other food to be consumed by an infant. Alternatively, the components may be provided in a solution form suitable to be immediately or upon dilution with a suitable solution consumed by the infant. The preceding solutions may be administered to the infant via a nasogastric or an orogastric route.

Examples of suitable dietary products include water, saline, buffered solutions, infant formula, and expressed breast milk, other suitable carriers, or combinations thereof. Any solution suitable for oral administration may be used. Additives may be added which act as bystander proteins (ie nonactive protein “filler”), which protects the EGF from enzymatic degradation by pancreatic proteases (42). For example, casein (a milk protein) has been used for this experimentally (42). Other approaches may involve administering with a protease inhibitor to preserve EGF structure and activity.

Alternatively, a treatment of the invention may be administered orally, enterally, parenterally, intravenously, subcutaneously, nasally or by enema. It may be possible to choose an intravenous route for an initial period of treatment as, for example, when initially treating NEC, during which period some patients are incapable of receiving an orally administered treatment. At such time, the patient typically has ileus. Ileus is typically diagnosed on physical examination by a lack of normal bowel sounds and by feed intolerance and vomiting. Typical time course is one to two weeks. Typically oral feeding is begun 10 days following diagnosis of ileus, and resumption of normal bowel sounds and resumption of feed tolerance can be confirmed if thought necessary. Once the ileus is resolved, treatment may be continued through a second period in which the treatment is administered orally. The composition may be prepared as a spray, solution, suspension, colloid, concentrate, powder, granules, tablets, pressed tablets, capsules (included coated and uncoated tablets or capsules), suppository and the like. Delayed release or controlled release formulations are also included.

The formulations may include additives such as viscosity adjusting agents, osmosity adjusting agents, buffers, pH adjusting agents, flavorings, stabilizers, colorings, preservatives and the like where required.

A unit dose would be a dosage suitable for administration in a single administration, i.e., a single feeding of an infant. A unit dose thus includes from about 200 mg/kg/day (0.9 mmol/kg/day) to about 500 mg/kg/day (2.4 mmol/kg/day) of L-arginine, or more preferably from about 250 mg/kg/day (1.2 mmol/kg/day) to about 400 mg/kg/day (1.9 mmol/kg/day), or more preferably from about 300 mg/kg/day (1.4 mmol/kg/day) to about 350 mg/kg/day (1.6 mmol/kg/day) L-arginine and from about 0.2 ug/kg/day (0.032 nmol/kg/day) to about 2 mg/kg/day (0.32 umol/kg/day), or more preferably from about 1 ug/kg/day (0.16 nmol/kg/day) to about 1 mg/kg/day (0.16 mmol/kg/day), or more preferably from about 2 ug/kg/day (0.32 nmol/kg/day) to about 0.2 mg/kg/day (32 nmol/kg/day) of EGF receptor agonist. In this specification “ug” means microgram, “umol” means micromole, etc. Typically, the L-arginine-EGF receptor agonist ratio would be between about 1:45400000 mol EGF receptor agonist/mol L-arginine and about 1:4500 mol EGF receptor agonist/mol L-arginine, more likely between 1:4540000 mol EGF receptor agonist/mol L-arginine and 1:45000 mol EGF receptor agonist/mol L-arginine. It is contemplated that treatment would be administered probably at least once a day, 3 or 4 times per day, or even continuously. Intermittent doses could be administered by any convenient route, e.g., bolus infusion, oral preparations discussed elsewhere herein, etc., subcutaneously, or by continuous IV drip. More continual administration would more typically be by I.V. drip or controlled release implants.

Generally speaking, EGF is prepared by a synthetic process, being manufactured by conventional biotechnological or chemical techniques. Of course, EGF might be obtained from a natural source.

Preferably, the combination of factors of the invention would be provided as a single mixture and administered together, but they could be provided in a kit in separate compartments and mixed for administration, or administered separately. Preferably, both are biologically available to the luminal side of epithelial cells of the intestines.

When prepared for mixture with a liquid, as for delivery with water or infant formula, the unit dose could have a solubility enhancer incorporated thereinto.

It may well be that the effectiveness of a dosage would be increased by use of a coated composition, one that would not dissolve until it reached the intestine. Reference may be made to “Remington's Pharmaceutical Sciences”, edited by Gennaro (Mack Publishing Company, 19th Ed., 1995). Pharmaceutically acceptable salts of the active agents (e.g., acid addition salts of L-arginine) may be prepared using standard procedures known to those skilled in the art of synthetic organic chemistry (34). As well, it may be desirable to include a suitable pharmaceutically acceptable carrier such as those used conventionally with peptide-based drugs, such as diluents, excipients and the like.

The product would of course be provided in sealed sterile packaging. Typically the EGF or equivalent polypeptide is provided as a lyophilized material.

The concurrent administration of L-arginine with EGF produces a synergistic benefit in the treatment and prevention of NEC. In a study by Dvorak et al. (48) no significant clinical benefit was reported when EGF was administered at a physiological concentration of 100 ng/ml. In these studies EGF at a concentration of 500 ng/ml was used to demonstrate a protective effect. The results of the studies related to the present invention showed that 100 ng/ml of EGF administered in combination with 2.7 mg/ml L-arginine provided significant clinical benefit in the prevention and treatment of NEC. Furthermore, the combination of EGF and L-arginine provided significant benefit over that seen when 100 ng/ml EGF or 2.7 mg/ml L-arginine were individually administered.

### REFERENCES

1. Akisu, M., Ozmen, D., Baka, M., Habif, S., Yalaz, M., Arslanoglu, S., Kultursay, N., Bayindir, O. Protective effect of dietary supplementation with L-arginine and L-carnitine on hypoxia/reoxygenation-induced necrotizing enterocolitis in young mice. Biol. Neonate 81: 260-5, 2002.

2. Amin, H. J., Zamora, S. A., McMillan, D. D., Fick, G. H., Butzner, J. D., Parsons, H. G., and Scott, R. B. Arginine supplementation prevents necrotizing enterocolitis in the preterm infant. Journal of Pediatrics 140, 425-431, 2002.

3. Boccia, D., Stolfi, I., Lana, S., and Moro, M. L. Nosocomial necrotizing enterocolitis outbreaks: epidemiology and control measures. Eur. J. Pediatr. 160, 385-391. 2001.

4. Ng, S. Necrotizing enterocolitis in the full term infant. J. Pediatr. Child Health 37: 1-4, 2001.

b 5. Bradford, M. M. A rapid and sensitive method for the quantitation of microgram quantities of protein utilizing the principle of protein-dye binding. Anal. Biochem. 72: 248-254, 1976.

6. Buret, A., Gall, D. G., Olson, M. E., and Hardin, J. A. Epidermal growth factor (EGF) prevents Escherichia Coli enteritis in rabbits and bacterial translocation in vitro. 7th International Congress for Infectious Disease, 1996.

7. Buret, A., Gall, D. G., Olson, M. E., and Hardin, J. A. Prevention of enteric infections by epidermal growth factor (EGF). 36th Interscience Conference on Antimicrobial Agents and Chemotherapy, New Orleans, La., 1996.

8. Buret, A., Gall, D. G., Olson, M. E., and Hardin, J. A. Anti-infective properties of a mucosal cytokine: epidermal growth factor (EGF). Proceedings of the International Biofilm Symposium, Canmore, Alberta, 1997.

9. Buret, A., Hardin, J. A., Olson, M. E., Chin, A., and Gall, D. G. Effects of orally administered epidermal growth factor (EGF) during Escherichia coli infection in rabbits. Gastroenterology 110, A793, 1996.

10. Buret, A., Kamieniecky, D., Olson, M. E., Gall, D. G., and Hardin, J. A. Epithelial colonization by Cryptosporidium and trans-epithelial electrical resistance: effects of epidermal growth factor (EGF). Gastroenterology 116, A865, 1999.

11. Buret, A., M. E. Olson, D. G. Gall, and J. A. Hardin. Effects of orally administered epidermal growth factor on enteropathogenic Escherichia coli infection in rabbits. Infect. Immun. 66: 4917-4923, 1998.

12. Buret, A., Olson, M. E., Gall, D. G., Hardin, J. A., Kamieniecky, D., and Lupul, S. Epidermal growth factor (EGF) inhibits intestinal colonization with Cryptosporidium parvum. Arch. Pharmacol. 358 (Suppl. 1), 8359, 1998.

13. Bury, R. G. and Tudehope, D. Enteral antibiotics for preventing necrotizing enterocolitis in low birthweight or preterm infants (Cochrane Review). The Cochrane Library Issue 1. 2001. Oxford: Update Software.

14. Caplan, M. S., Miller-Catchpole, R., Kaup, S., Russell, T., Lickerman, M., Amer, M., Xiao, Y., Thomson, R., Jr. Bifidobacterial supplementation reduces the incidence of necrotizing enterocolitis in a neonatal rat model. Gastroenterology 117: 577-83, 1999.

15. Caplan, M. S., Hedlund, E., Adler, L., Lickerman, M., Hsueh, W. The platelet-activating factor receptor antagonist WEB 2170 prevents neonatal necrotizing enterocolitis in rats. J. Pediatr. Gastroenterol. Nutr. 24: 296-301, 1997.

16. Caplan, M. S., B. Hedlund, N. Hill, and W. MacKendrick. The role of endogenous nitric oxide and platelet-activating factor in hypoxia-induced intestinal injury in rats. Gastroenterology 106: 346-352, 1994.

17. Caplan, M. S. and T. Jilling. New concepts in necrotizing enterocolitis. Curr. Opin. Pediatr. 13: 111-115, 2001.

18. Caplan, M. S., R. Miller-Catchpole, S. Kaup, T. Russell, M. Lickerman, M. Amer, Y. Xiao, R. Thomson, Jr., and. Bifidobacterial supplementation reduces the incidence of necrotizing enterocolitis in a neonatal rat model. Gastroenterology 117: 577-583, 1999.

19. Carlson, S. E., Montalto, M. B., Ponder, D. L., Werkman, S. H., Korones, S. B. Lower incidence of necrotizing enterocolitis in infants fed a preterm formula with egg phospholipids. Pediatr. Res. 44: 491-98, 1998.

20. Claud, E. C. and Walker, W. A. Hypothesis: inappropriate colonization of the premature intestine can cause neonatal necrotizing enterocolitis. FASEB J. 15, 1398-1403. 2001.

21. Dahlquist, A. Method for assay of intestinal dissacharidases. Anal. Biochem. 7: 1825, 1964.

22. Di Lorenzo, M., J. Bass, and A. Krantis. Use of L-arginine in the treatment of experimental necrotizing enterocolitis. J. Pediatr. Surg. 30: 235-241, 1995.

23. Dvorák, B., Halpern, M. D., Holubec, H., Dvorakova, K., Dominguez, J. A., Williams, C. S., Meza, Y. G., Kozakova, H., McCluskey, R. S. Maternal milk reduces the severity of necrotizing enterocolitis and increases intestinal IL-10 in a neonatal rat model. Pediatr. Res. 53: 426-33, 2003.

24. Eibl, M. M., Wolf, H. M., Furnkranz, H., Rosenkranz, A. Prevention of necrotizing enterocolitis in low-birth-weight infants by IgA-IgG feeding. N. Engl. J. Med. 319: 1-7, 1988.

25. Elliott, S. N., McKnight, W., Gall, D. G., Hardin, J. A., Olson, M. E., Wallace, J. L., and Buret, A. Epidermal growth factor (EGF)—induced gastric ulcer healing is independent of a bactericidal action. Mediators of Inflammation 8 (Suppl. 1): S81, 1999.

26. Elliott, S. N., J. L. Wallace, W. McKnight, D. G. Gall, J. A. Hardin, M. Olson, and A. Buret. Bacterial colonization and healing of gastric ulcers: the effects of epidermal growth factor. Am. J. Physiol. Gastrointest. Liver Physiol 278: 6105-6112, 2000.

27. Gibbs, S., A. N. Silva Pinto, S. Murli, M. Huber, D. Hohl, and M. Ponec. Epidermal growth factor and keratinocyte growth factor differentially regulate epidermal migration, growth, and differentiation. Wound Repair Regen. 8: 192-203, 2000.

28. Halac, E., Halac, J., Begue, E. F., Casanas, J. M., Indiveri, D. R., Petit, J. F., Figueroa, M. J., Olmas, J. M., Rodriguez, L. A., Obregon, R. J. Prenatal and postnatal corticosteroid therapy to prevent necrotizing enterocolitis: a controlled trial. J. Pediatr. 117: 132-8, 1990.

29. Hardin, J. A., Olson, M. E., Buret, A. G., and Gall, D. G. The effect of oral EGF on Giardiasis in gerbils. Gastroenterology 112, A992, 1997.

30. Hentschel, J., de Veer, I., Gastmeier, P., Ruden, H., and Obladen, M. Neonatal nosocomial infection surveillance: incidences by site and a cluster of necrotizing enterocolitis. Infection 27: 234-238, 1999.

31. Hinegardner, R. An improved fluorometric assay for DNA. Anal. Biochem. 39: 197201, 1971.

32. Hoyos, A. B. Reduced incidence of necrotizing enterocolitis associated with enteral administration of Lactobacillus acidophilus and Bifidobacterium infantis to neonates in an intensive care unit. Int. J. Infect. Dis. 3: 197-202, 1999.

33. Hui, W. M., B. W. Chen, A. W. C. Kung, C. H. Cho, C. T. Luk, and S. K. Lam. Effect of epidermal growth factor on gastric blood flow in rats: possible role in mucosal protection. Gastroenterology 104: 1605-1610, 1993.

34. March, J. “Advanced Organic Chemistry: Reactions, Mechanisms and Structure”, 4th Ed. (New York: Wiley-Interscience) 1992.

35. Neu, J. Necrotizing enterocolitis: the search for a unifying pathogenic theory leading to prevention. Pediatr. Clin. North Am. 43: 409-432, 1996.

36. Kubes, P. Ischemia-reperfusion in feline small intestine: a role for nitric oxide. Am. J. Physiol. Gastrointest. Liver Physiol 264: 6143-6149, 1993.

37. McGuire, W., and Anthony, M. Y. Donor human milk versus formula for preventing necrotizing enterocolitis in preterm infants: systematic review. Arch. Dis. Child Fetal Neonatal Ed. 88: F11-4, 2003.

38. Opleta-Madsen, K., J. B. Meddings, and D. G. Gall. Epidermal growth factor and postnatal development of intestinal transport and membrane structure. Pediatr. Res. 30: 342-350, 1991.

39. Opleta, K., E. V. O'Loughlin, E. A. Shaffer, J. Hayden, M. D. Hollenberg, and D. G. Gall. Effect of epidermal growth factor on growth and postnatal development of the rabbit liver. Am. J. Physiol. Gastrointest. Liver Physiol. 253: 6622-6626, 1987.

40. Ozturk, H., Dokucu, A. I., Ogun, C., Buyukbayram, H. Protective effects of recombinant human interleukin-10 on intestines of hypoxia-induced necrotizing enterocolitis in immature rats. J. Pediatr. Surg. 37: 1330-3, 2002.

41. Piazuelo, E., P. Jimenez, A. Lanas, A. Garcia, F. Esteva, and R. Sainz. Platelet derived growth factor and epidermal growth factor play a major role in human colonic fibroblast repair activities. Eur. Surg. Res. 32: 191-196, 2000.

42. Playford, R. J., Woodman, A. C., Clark, P., Watanapa, P., Vesey, D., Deprez, P. H., Williamson, R. C. N., Calam, J. Effect of luminal growth factor preservation on intestinal growth. Lancet 341: 843-8, 1993.

43. Schanler, R. J. The use of human milk for premature infants. Pediatr. Clin. North Am. 48: 207-219, 2001.

44. Shin, C. E., R. A. Falcone, Jr., L. Stuart, C. R. Erwin, and B. W. Warner. Diminished epidermal growth factor levels in infants with necrotizing enterocolitis. J. Pediatr. Surg. 35: 173-176, 2000.

45. Siu, Y. K., Ng, P. C., Fung, S. C., Lee, C. H ., Wong, M. Y., Fok, T. F., So, K. W., Cheung, K. L., Wong, W., Cheng, A. F. Double blind, randomized, placebo controlled study of oral vancomycin in prevention of necrotizing enterocolitis in preterm, very low birthweight infants. Arch. Dis. Child. Fetal Neonatal Ed. 79: F105-9, 1998.

46. Walsh, M. C., and Kliegman, R. M. Necrotizing enterocolitis: treatment based on staging criteria. Pediatr. Clin. North Am. 33: 179-201, 1986.

47. Zamora, S. A., Amin, H. J., McMillan, D. D., Kubes, P., Fick, G. H., Butzner, J. D., Parsons, H. G., and Scott, R. B. Plasma L-arginine concentrations in premature infants with necrotizing enterocolitis. J. Pediatr. 131, 226-232. 1997.

48. Dvorák, B.; Halpern, M. D.; Holubec, H.; Williams, C. S.; McWilliam, D. L.; Dominguez, J. A.; Stepankova, R.; Payne, C. M.; McCuskey, R. S. Epidermal growth factor reduces the development of necrotizing enterocolitis in a neonatal rat model. Am. J. Physiol Gastrointest. Liver Physiol 282: G156-G164, 2002.

49. Jorgensen, P. E., L. G. Jensen, B. S. Sorensen, S. S. Poulsen, and E. Nexo. Pig epidermal growth factor precursor contains segments that are highly conserved among species. Scand. J. Clin. Lab. Invest. 58: 287-297, 1998.

50. Nexo, E. and H. F. Hansen. Binding of epidermal growth factor from man, rat and mouse to the human epidermal growth factor receptor. Biochim. Biophys. Acta 843: 101-106, 1985.

51. Pascall, J. C., D. S. C. Jones, S. M. Doel, J. M. Clements, M. Hunter, T. Fallon, M. Edwards, and K. D. Brown. Cloning and characterization of a gene encoding pig epidermal growth factor. J. Mol. Endocrinol. 6: 63-70, 1991

52. Simpson, R. J., J. A. Smith, R. L. Moritz, M. J. O'Hare, P. S. Rudland, J. R. Morrison, C. J. Lloyd, B. Grego, A. W. Burgess, and E. C. Nice. Rat epidermal growth factor: complete amino acid sequence. Eur. J. Biochem. 153: 629-637, 1985.

53. Lenferink, A. E., E. J. Van Zoelen, M. J. Van Vugt, S. Grothe, W. van Rotterdam, M. L. Van de Poll, and M. D. O'Connor-McCourt. Superagonistic activation of ErbB-1 by EGF-related growth factors with enhanced association and dissociation rate constants. J. Biol. Chem. 275: 26748-26753, 2000.

54. Marion, R., M. Coeffier, A. Leplingard, L. Favennec, P. Ducrotte, and P. Dechelotte. Cytokine-stimulated nitric oxide production and inducible NO-synthase mRNA level in human intestinal cells: lack of modulation by glutamine. Clin. Nutr. 22: 523-528, 2003.

55. Kikuchi, K., T. Nagano, H. Hayakawa, Y. Hirata, and M. Hirobe. Detection of nitric oxide production from a perfused organ by a luminol-H₂O₂ system. Anal. Chem. 65: 1794-1799, 1993.

56. Kojima, H., N. Nakatsubo, K. Kikuchi, S. Kawahara, Y. Kirino, H. Nagoshi, Y. Hirata, and T. Nagano. Detection and imaging of nitric oxide with novel fluorescent indicators: diaminoflouresceins. Anal. Chem. 70: 2446-2453, 1998.

57. Snygg, J., A. Aneman, A. Pettersson, and L. Fandriks. Intestinal nitric oxide output during reduced mucosal blood flow in healthy volunteers. Crit. Care Med. 31: 2198-2204, 2003.

58. Levine, D. Z., M. Iacovitti, K. D. Bums, and X. Zhang. Real-time profiling of kidney tubular fluid nitric oxide concentrations in vivo. Am. J. Physiol. Renal Physiol. 281: F189-F194, 2001.

59. Boger, R. H. and S. M. Bode-Boger. The clinical pharmacology of L-arginine. Ann. Rev. Pharmacol. Toxicol. 41: 79-99, 2001.

60. Feelisch, M. The use of nitric oxide donors in pharmacological studies. Naunyn Schmiedebergs Arch. Pharmacol. 358: 113-122, 1998.

61. Pacher, P., J. G. Mabley, L. Liaudet, O. V. Evgenov, G. J. Southan, G. E. Abdelkarim, C. Szabo, and A. L. Salzman. Topical administration of a novel nitric oxide donor, linear polyethylenimine-nitric oxide/nucleophile adduct (DS1), selectively increases vaginal blood flow in anesthetized rats. Int. J. Impot. Res. 15: 461-464, 2003.

62. Zell, R., R. Markgraf, M. Schmidtke, M. Gorlach, A. Stelzner, A. Henke, H. H. Sigusch, and B. Gluck. Nitric oxide donors inhibit the coxsackievirus B3 proteinases 2A abd 3C in vitro, virus production in cells, and signs of myocarditis in virus-infected mice. Med. Microbiol. Immunol. 193: 91-100, 2004.

1. A composition comprising an EGF receptor agonist and L-arginine, a bioequivalent of L-arginine, or an NO-donor wherein the ratio of the agonist and L-arginine, bioequivalent thereof, or NO-donor is between 1:45,000,000 and 1:4,500 (mole:mole), or between 1:20,000,000 and 1:100,000, or between 1:10,000,000 and 1:1,000,000, or between 1:4,700,000 and 1:47,000.

2. The composition of claim 1 in solid form.

3. The composition of claim 1 or 2 in lyophilized form.

4. The composition of claim 1 in solution form.

5. The composition of claim 4, wherein the solution is suitable for oral delivery to a human.

6. The composition of claim 4, wherein the solution is suitable for enteral delivery to a human.

7. The composition of any of claims 1 to 6, wherein the EGF receptor agonist is EGF.

8. The composition of claim 7, wherein the EGF receptor agonist is synthetic, optionally manufactured by chemical synthesis or recombinantly, or derived from natural sources.

9. The composition of any of claims 1 to 8, for the treatment of necrotizing enterocolitis.

10. A unit dose comprising L-arginine, bioequivalent thereof, or an NO-donor and an EGF receptor agonist suitable for the oral administration to an animal upon dissolution with a pharmaceutically acceptable liquid.

11. The unit dose of claim 10, wherein the pharmaceutically acceptable liquid is selected from the group consisting of water, saline, infant formula, buffered solution, expressed breast milk, other suitable carriers, and combinations thereof.

12. The unit dose of claim 10 or 11, comprising L-arginine in a quantity of from about 200 mg/kg/day (0.9 mmol/kg/day) to about 500 mg/kg/day (2.4 mmol/kg/day), or more preferably from about 250 mg/kg/day (1.2 mmol/kg/day) to about 400 mg/kg/day (1.9 mmol/kg/day), or more preferably from about 300 mg/kg/day (1.4 mmol/kg/day) to about 350 mg/kg/day (1.6 mmol/kg/day).

13. The unit dose of claims 10, 11, or 12, wherein the EGF receptor agonist is supplied in a quantity of 0.032 nmol/kg/day to about 0.32 umol/kg/day, or more preferably from about 0.16 nmol/kg/day to about 0.16 mmol/kg/day, or more preferably from about 0.32 nmol/kg/day to about 32 nmol/kg/day.

14. The unit dose of any of claim 10, 11, 12, or 13, wherein the EGF receptor agonist is EGF.

15. The unit dose of any of claim 10, 11, 12, 13, or 14, wherein the animal is a human infant.

16. The unit dose of claim 15 wherein the human infant is premature.

17. A unit dose comprising L-arginine, a bioequivalent thereof, or an NO-donor and an EGF receptor agonist suitable for the intravenous administration to a human infant, optionally upon dissolution with a pharmaceutically suitable solution.

18. The unit dose of claim 17, wherein the pharmaceutically acceptable solution is saline or a buffered solution.

19. The unit dose of claims 17 or 18, wherein the ratio of the agonist and L-arginine, bioequivalent thereof, or NO-donor is between 1:45,000,000 and 1:4,500 (mole:mole), or between 1:20,000,000 and 1:100,000, or between 1:10,000,000 and 1:1,000,000, or between 1:4,700,000 and 1:47,000.

20. The unit dose of any of claims 17, 18, or 19, wherein the EGF receptor agonist is EGF.

21. The unit dose of any of claim 17, 18, 19, or 20, wherein the animal is a human infant.

22. The unit dose of claim 21 wherein the human infant is premature.

23. A method of treating necrotizing enterocolitis in an animal, the method comprising administering an EGF receptor agonist and L-arginine, a bioequivalent thereof, or an NO-donor to the animal.

24. A method of prophylaxis of necrotizing enterocolitis in an animal, the method comprising administering an EGF receptor agonist and L-arginine, a bioequivalent thereof, or an NO-donor to the animal at risk for necrotizing enterocolitis.

25. The method of claim 24 wherein the animal is an infant, particularly an infant suffering from cardiovascular disturbances, or a premature infant at an age prior to normal term.

26. The method of claim 25 wherein the animal is a human infant having a weight of less than or equal to about 1700 g, or less than about 1400 g, more preferably less than about 1300 g, more preferably less than about 1200 g, more preferably less than about 1100 g, more preferably less than about 1000 g, more preferably less than about 900 g, more preferably less than about 800 g, more preferably less than about 750 g.

27. A method of treating necrotizing enterocolitis or of prophylaxis of necrotizing enterocolitis in a premature infant, the method comprising enterally administering to the infant an EGF receptor agonist and L-arginine, a bioequivalent thereof, or an NO-donor.

28. The method of claim 27, wherein the EGF receptor agonist and L-arginine, a bioequivalent thereof, or an NO-donor are administered together in a mixture.

29. The method of claim 28, wherein the mixture is administered at least once daily.

30. The method of any of claims 27 to 29, wherein the EGF receptor agonist is EGF.

31. The method of claim 30, wherein the EGF is synthesized recombinantly.

32. A kit comprising therapeutic amounts of an EGF receptor agonist and L-arginine, a bioequivalent thereof, or an NO-donor, and instructions for use in the treatment of a medical disorder.

33. The kit of claim 32, wherein the medical disorder is necrotizing enterocolitis.

34. The kit of claim 32, wherein the agonist and L-arginine, a bioequivalent thereof, or NO-donor are supplied combined in solid form.

35. The kit of claim 34, wherein the instructions include the step of dissolving the solid form in a solution suitable for oral administration.

36. The kit of claim 34, wherein the instructions include the step of dissolving the solid form in a solution suitable for intravenous administration.

37. The kit of claim 34, wherein the agonist and L-arginine, a bioequivalent thereof, or an NO-donor are supplied separately.

38. The kit of claim 37, wherein at least one of the agonist and L-arginine, a bioequivalent thereof, or NO-donor is in solid form.

39. The kit of claim 37, wherein at least one of the agonist and L-arginine, a bioequivalent thereof, or NO-donor is in solution.

40. The kit of any of claims 37 to 39, wherein the instructions include a step of mixing the agonist and L-arginine, a bioequivalent thereof, or an NO-donor before administration.

41. A method of treating NEC in an animal comprising delivering an EGF receptor agonist to the intestinal tract of the animal and increasing the in vivo generation of NO within the intestinal tract of the animal.

42. The method of claim 41, wherein increasing the in vivo generation of NO includes administering a substrate of nitric oxide synthase, or a bioequivalent of the substrate, to the animal.

43. The method of claim 41, wherein increasing the in vivo generation of NO includes administering an NO-donor.

44. A method of treating a person, optionally an infant, at risk of NEC comprising delivering an EGF receptor agonist to the intestinal tract of the patient and increasing the in vivo generation of NO within the intestinal tract of the person.

45. The method of claim 44, wherein increasing the in vivo generation of NO includes administering a substrate of nitric oxide synthase, or a bioequivalent of the substrate, to the person.

46. The method of claim 44, wherein increasing the in vivo generation of NO includes administering an NO-donor.

47-60. (canceled)
